(19) United States
(12) Patent Application Publication
(10) Pub. No.: US 2008/0274882 A1
(43) Pub. Date: Nov. 6, 2008

(54) Insecticides Based on Selected Neonicotinoids and Strobilurins

(75) Inventors: Peter-Wilhelm Krohn, Leverkusen (DE); Rolf Christian Becker, Burscheid (DE); Heike Hungenberg, Langenfeld (DE)

Correspondence Address: STERNE, KESSLER, GOLDSTEIN & FOX P.L.L.C., 1100 NEW YORK AVENUE, N.W., WASHINGTON, DC, 20005, US

(73) Assignee: Bayer CropScience AG, Monheim (DE)

(21) Appl. No.: 11/722,648

(22) PCT Filed: Dec. 17, 2005
(86) PCT No.: PCT/EP05/13622

§ 371 Date: Mar. 13, 2008

(30) Foreign Application Priority Data
Dec. 24, 2004 (DE) 102004062513.1

(51) Int. Cl. A01N 25/26; A01N 43/40; A61K 31/415; A01P 7/04; A01N 43/72

(52) U.S. Cl. 504/100; 514/341; 514/277; 514/342; 514/407; 514/357; 514/229.2

(57) ABSTRACT

The present invention relates to novel active compound combinations comprising, as active compounds, firstly an active compound from the group of the neonicotinoids clothianidin, imidacloprid, thiacloprid, dinotefuran, acetamiprid, nitenpyram and thiamethoxam and, secondly, at least one active compound selected from the group of the strobilurin fungicides picoxystrobin, pyraclostrobin, dimoxystrobin, metominostrobin and orysastrobin, to their use as pesticides and as seed dressings for the treatment of seed, to methods for controlling pests and for protecting seed and to seed treated with the active compound combinations according to the invention.

The present invention relates to novel active compound combinations comprising, as active compounds, firstly an active compound from the group of the neonicotinoids and, secondly, at least one active compound selected from the group of the strobilurin fungicides, which combinations have surprisingly good insecticidal and fungicidal properties.

It is known that the strobilurin fungicide picoxystrobin of the formula

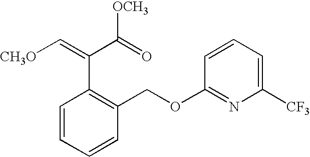

can be used as fungicide for controlling fungal diseases of the plant. Picoxystrobin is described, for example, in EP 0 278 595 A2 or “The Pesticide Manual”, 13th edition (2003), published by the British Crop Protection Council.

It is also known that the strobilurin fungicide pyraclostrobin of the formula

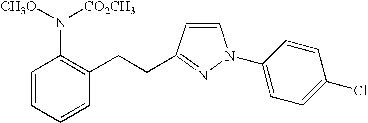

can be used as fungicide for controlling fungal diseases of the plant. Pyraclostrobin is described, for example, in “The Pesticide Manual”, 13th edition (2003), published by the British Crop Protection Council or else in WO 96/01256 A1.

It is also known that the strobilurin fungicide dimoxystrobin of the formula

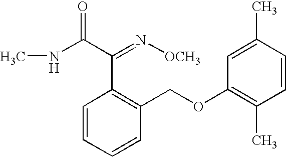

can be used as fungicide for controlling fungal diseases of the plant. Dimoxystrobin is described, for example, in EP 0 398 692 A2 or in “The Pesticide Manual”, 13th edition (2003), published by the British Crop Protection Council.

It is also already known that the strobilurin fungicide metominostrobin of the formula

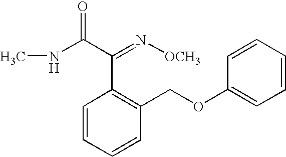

can be used as fungicide for controlling fungal diseases of the plant. Metaminostrobin is described, for example, in EP 0 398 692 A2 or in “The Pesticide Manual”, 13th edition (2003), published by the British Crop Protection Council.

It is also already known that the strobilurin fungicide orysastrobin of the formula

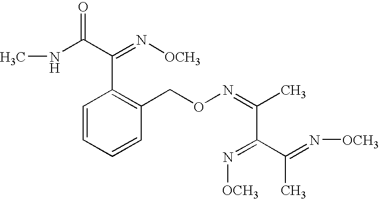

can be used as fungicide for controlling fungal diseases of the plant. Metaminostrobin is described, for example, in EP 0 876 332 A1.

In the fungal cells, the strobilurins mentioned block the electron transport in the respiratory chain, thus preventing the production of ATP.

Furthermore, it is known that neonicotinoids such as, for example, imidacloprid, thiacloprid, clothianidin, thiamethoxam, acetamiprid, nitenpyram and dinotefuran are suitable for controlling animal pests, in particular insects.

The activity of these compounds is good; however, at low application rates or against individual pests, it does sometimes not meet the high demands made on pesticides.

It has now been found that mixtures comprising at least one compound from the group of the neonicotinoids mentioned below and at least one of the strobilurins mentioned above from the group consisting of picoxystrobin, pyraclostrobin, dimoxystrobin and orysastrobin are synergistically active and particularly suitable for controlling animal pests and fungi, the respective activity of this combination being higher than the activity of the individual active compounds. By using these mixtures according to the invention, it is possible to use considerably lower amounts of active compounds, i.e. the activity of the mixture is greater than the activity of the individual components.

Surprisingly, it was also found that the combinations according to the invention are particularly suitable for treating seed and protecting the plants emerging therefrom against pests and attack by fungi. In this context, particular mention may be made of mixtures comprising at least one neonicotinoid from the group consisting of clothianidin and imidacloprid, with clothiandin being specially preferred. Preference is also given to mixtures comprising at least one strobilurin from the group consisting of picoxystrobin, pyraclostrobin and dimoxystrobin.

The neonicotinoids mentioned above are known, for example from “The Pesticide Manual”, 13th edition (2003), published by the British Crop Protection Council. Clothianidin has the formula

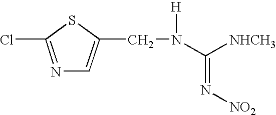

and is known from EP A2 0 376 279.

Thiacloprid has the formula

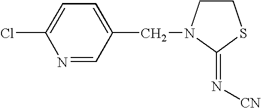

and is known from EP A2 0 235 725.

Dinotefuran has the formula

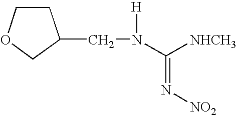

and is known from EP A1 0 649 845.

Acetamiprid has the formula

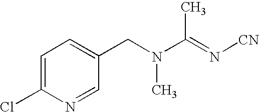

and is known from WO A1 91/04965.

Nitenpyram has the formula

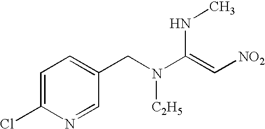

and is known from EP A2 0 302 389.

Imidacloprid has the formula

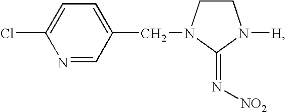

and is known from EP A1 0 192 060.

Thiamethoxam has the formula

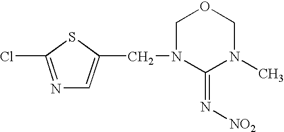

and is known from EP A2 0 580 553.

The ratio of the active compounds employed to one another and the total amount of the mixture to be applied depends on the nature and the occurrence of the insects and fungi. For each application, the optimum ratios and total amounts applied can in each case be determined by test series. In general, the weight ratio of an active compound selected from the strobilurins mentioned above and an active compound selected from one of the neonicotinoids mentioned above is between 1000:1 and 1:100, preferably between 625:1 and 1:100, particularly preferably between 125:1 and 1:50 and very particularly preferably between 25:1 and 1:5, where in the ratios here the fungicide is mentioned first.

A particularly preferred mixture according to the invention comprises the active compound picoxystrobin and clothianidin. In the mixture, the weight ratio of the two active compounds to one another is between 1000:1 and 1:100, preferably between 625:1 and 1:100, particularly preferably between 125:1 and 1:50 and very particularly preferably between 25:1 and 1:5, where in the ratios here and below picoxystrobin is in each case mentioned first.

A further particularly preferred mixture according to the invention comprises the active compound pyraclostrobin and clothianidin. In the mixture, the weight ratio of the two active compounds to one another is between 1000:1 and 1:100, preferably between 625:1 and 1:100, particularly preferably between 125:1 and 1:50 and very particularly preferably between 25:1 and 1:5, where in the ratios here and below pyraclostrobin is in each case mentioned first.

A further particularly preferred mixture according to the invention comprises the active compound dimoxystrobin and clothianidin. In the mixture, the weight ratio of the two active compounds to one another is between 1000:1 and 1:100, preferably between 625:1 and 1:100, particularly preferably between 125:1 and 1:50 and very particularly preferably between 25:1 and 1:5, where in the ratios here and below dimoxystrobin is in each case mentioned first.

A particularly preferred mixture according to the invention comprises the active compound metominostrobin and clothianidin. In the mixture, the weight ratio of the two active compounds to one another is between 1000:1 and 1:100, preferably between 625:1 and 1:100, particularly preferably between 125:1 and 1:50 and very particularly preferably between 25:1 and 1:2, where in the ratios here and below metominostrobin is in each case mentioned first.

A further particularly preferred mixture according to the invention comprises the active compound orysastrobin and clothianidin. In the mixture, the weight ratio of the two active compounds to one another is between 1000:1 and 1:100, preferably between 625:1 and 1:100, particularly preferably between 125:1 and 1:50 and very particularly preferably between 25:1 and 1:5, where in the ratios here and below orysastrobin is in each case mentioned first.

A particularly preferred mixture according to the invention comprises the active compound picoxystrobin and imidacloprid. In the mixture, the weight ratio of the two active compounds to one another is between 1000:1 and 1:100, preferably between 625:1 and 1:100, particularly preferably between 125:1 and 1:50 and very particularly preferably between 25:1 and 1:5, where in the ratios here and below picoxystrobin is in each case mentioned first.

A further particularly preferred mixture according to the invention comprises the active compound pyraclostrobin and imidacloprid. In the mixture, the weight ratio of the two active compounds to one another is between 1000:1 and 1:100, preferably between 625:1 and 1:100, particularly preferably between 125:1 and 1:50 and very particularly preferably between 25:1 and 1:5, where in the ratios here and below pyraclostrobin is in each case mentioned first.

A further particularly preferred mixture according to the invention comprises the active compound dimoxystrobin and imidacloprid. In the mixture, the weight ratio of the two active compounds to one another is between 1000:1 and 1:100, preferably between 625:1 and 1:100, particularly preferably between 125:1 and 1:50 and very particularly preferably between 25:1 and 1:5, where in the ratios here and below dimoxystrobin is in each case mentioned first.

A particularly preferred mixture according to the invention comprises the active compound metominostrobin and imidacloprid. In the mixture, the weight ratio of the two active compounds to one another is between 1000:1 and 1:100, preferably between 625:1 and 1:100, particularly preferably between 125:1 and 1:50 and very particularly preferably between 25:1 and 1:5, where in the ratios here and below metominostrobin is in each case mentioned first.

A further particularly preferred mixture according to the invention comprises the active compound oryastrobin and imidacloprid. In the mixture, the weight ratio of the two active compounds to one another is between 1000:1 and 1:100, preferably between 625:1 and 1:100, particularly preferably between 125:1 and 1:50 and very particularly preferably between 25:1 and 1:5, where in the ratios here and below orysastrobin is in each case mentioned first.

Preferred mixtures in the sense of the present invention also include combinations of thiacloprid and one of the strobilurin fungicides from the group consisting of picoxystrobin, pyraclostrobin, dimoxystrobin, metominostrobin and orysastrobin.

Preferred mixtures in the sense of the present invention also include combinations of dinotefuran and one of the strobilurin fungicides from the group consisting of picoxystrobin, pyraclostrobin, dimoxystrobin, metominostrobin and orysastrobin.

Preferred mixtures in the sense of the present invention also include combinations of thiamethoxam and one of the strobilurin fungicides from the group consisting of picoxystrobin, pyraclostrobin, dimoxystrobin, metominostrobin and orysastrobin.

Preferred mixtures in the sense of the present invention also include combinations of nitenpyram and one of the strobilurin fungicides from the group consisting of picoxystrobin, pyraclostrobin, dimoxystrobin, metominostrobin and orysastrobin.

Preferred mixtures in the sense of the present invention also include combinations of acetamiprid and one of the strobilurin fungicides from the group consisting of picoxystrobin, pyraclostrobin, dimoxystrobin, metominostrobin and orysastrobin.

Preferred mixtures in the sense of the present invention also include combinations of two of the neonicotinoids mentioned above and one of the strobilurin fungicides from the group consisting of picoxystrobin, pyraclostrobin, dimoxystrobin, metominostrobin and orysastrobin. Here, mixtures comprising clothianidin and imidacloprid and also a strobilurin fungicide from the group consisting of picoxystrobin, pyraclostrobin, dimoxystrobin and orysastrobin may be mentioned as being preferred. The combinations of, firstly, clothianidin and thiacloprid or imidacloprid and thiacloprid and, secondly, a strobilurin fungicide from the group consisting of picoxystrobin, pyraclostrobin, dimoxystrobin, metominostrobin and orysastrobin may also be mentioned as being preferred.

Preferred mixtures in the sense of the present invention also include combinations of two of the strobilurin fungicides mentioned above from the group consisting of picoxystrobin, pyraclostrobin, dimoxystrobin and orysastrobin and one of the neonicotinoids mentioned above. Combinations of in each case, firstly, pyraclostrobin and picoxystrobin, pyraclostrobin and dimoxystrobin, or picoxystrobin and dimoxystrobin and, secondly, a neonicotinoid from the group consisting of clothianidin, imidacloprid, thiacloprid, dinotefuran, acetamiprid, nitenpyram and thiamethoxam may be mentioned as being preferred.

The preferred active compound combinations according to the invention are summarized in the table below:

| Active compound | Active compound of | Weight ratio of active compound |
|---|---|---|
| of group (a) | group (b) | of group (a) to active compound |
| -Neonicotinyls- | -Strobilurins- | of group (b) |
| clothianidin | picoxystrobin | 1000:1 to 1:100 |
| imidacloprid | picoxystrobin | 1000:1 to 1:100 |
| thiacloprid | picoxystrobin | 1000:1 to 1:100 |
| nitenpyram | picoxystrobin | 1000:1 to 1:100 |
| acetamiprid | picoxystrobin | 1000:1 to 1:100 |
| dinotefuran | picoxystrobin | 1000:1 to 1:100 |
| thiamethoxam | picoxystrobin | 1000:1 to 1:100 |
| clothianidin | pyraclostrobin | 1000:1 to 1:100 |
| imidacloprid | pyraclostrobin | 1000:1 to 1:100 |
| thiacloprid | pyraclostrobin | 1000:1 to 1:100 |
| nitenpyram | pyraclostrobin | 1000:1 to 1:100 |
| acetamiprid | pyraclostrobin | 1000:1 to 1:100 |
| dinotefuran | pyraclostrobin | 1000:1 to 1:100 |
| thiamethoxam | pyraclostrobin | 1000:1 to 1:100 |
| clothianidin | dimoxystrobin | 1000:1 to 1:100 |
| imidacloprid | dimoxystrobin | 1000:1 to 1:100 |
| thiacloprid | dimoxystrobin | 1000:1 to 1:100 |
| nitenpyram | dimoxystrobin | 1000:1 to 1:100 |
| acetamiprid | dimoxystrobin | 1000:1 to 1:100 |
| dinotefuran | dimoxystrobin | 1000:1 to 1:100 |
| thiamethoxam | dimoxystrobin | 1000:1 to 1:100 |
| clothianidin | orysastrobin | 1000:1 to 1:100 |
| imidacloprid | orysastrobin | 1000:1 to 1:100 |
| thiacloprid | orysastrobin | 1000:1 to 1:100 |
| nitenpyram | orysastrobin | 1000:1 to 1:100 |
| acetamiprid | orysastrobin | 1000:1 to 1:100 |
| dinotefuran | orysastrobin | 1000:1 to 1:100 |
| thiamethoxam | orysastrobin | 1000:1 to 1:100 |
| clothianidin | metominostrobin | 1000:1 to 1:100 |
| imidacloprid | metominostrobin | 1000:1 to 1:100 |
| thiacloprid | metominostrobin | 1000:1 to 1:100 |
| nitenpyram | metominostrobin | 1000:1 to 1:100 |
| acetamiprid | metominostrobin | 1000:1 to 1:100 |
| dinotefuran | metominostrobin | 1000:1 to 1:100 |

The active compound combinations, having good plant compatibility and favorable homeotherm toxicity, are suitable for controlling animal pests, in particular insects, arachnids and nematodes, encountered in agriculture, in forests, in the protection of stored products and materials and in the hygiene sector. They are preferably used as crop protection compositions for foliar and soil treatment.

The mixtures according to the invention are also highly effective against phytopathogenic fungi. By way of example, but not by way of limitation, some pathogens of fungal diseases may be mentioned:

Xanthomonas species, such as, for example, Xanthomonas campestris pv. oryzae; Pseudomonas species, such as, for example, Pseudomonas syringae pv. lachrymans; Erwinia species, such as, for example, Erwinia amylovora; Pythium species, such as, for example, Pythium ultimum; Phytophthora species, such as, for example, Phytophthora infestans; Pseudoperonospora species, such as, for example, Pseudoperonospora humuli or Pseudoperonospora cubensis; Plasmopara species, such as, for example, Plasmopara viticola; Bremia species, such as, for example, Bremia lactucae; Peronospora species, such as, for example, Peronospora pisi or P. brassicae; Erysiphe species, such as, for example, Erysiphe graminis; Sphaerotheca species, such as, for example, Sphaerotheca fuliginea; Podosphaera species, such as, for example, Podosphaera leucotricha; Venturia species, such as, for example, Venturia inaequalis; Pyrenophora species, such as, for example, Pyrenophora teres or P. graminea (conidia form: Drechslera, syn: Helminthosporium); Cochliobolus species, such as, for example, Cochliobolus sativus (conidia form: Drechslera, syn: Helminthosporium); Uromyces species, such as, for example, Uromyces appendiculatus; Puccinia species, such as, for example, Puccinia recondita; Sclerotinia species, such as, for example, Sclerotinia sclerotiorum; Tilletia species, such as, for example, Tilletia caries; Ustilago species, such as, for example, Ustilago nuda or Ustilago avenae; Pellicularia species, such as, for example, Pellicularia sasakii; Pyricularia species, such as, for example, Pyricularia oryzae; Fusarium species, such as, for example, Fusarium culmorum; Botrytis species, such as, for example, Botrytis cinerea; Septoria species, such as, for example, Septoria nodorum; Leptosphaeria species, such as, for example, Leptosphaeria nodorum; Cercospora species, such as, for example, Cercospora canescens; Alternaria species, such as, for example, Alternaria brassicae; Pseudocercosporella species, such as, for example, Pseudocercosporella herpotrichoides.

At certain application rates, the active compound combinations according to the invention may also have a strengthening effect in plants. Accordingly, they are also suitable for mobilizing the defense system of the plant against attack by unwanted microorganisms. This may, if appropriate, be one of the reasons of the enhanced activity of the combinations according to the invention, for example against fungi.

Plant-strengthening (resistance-inducing) substances are to be understood as meaning, in the present context, those substances or combinations of substances which are capable of stimulating the defense system of plants in such a way that, when subsequently inoculated with unwanted microorganisms, the treated plants display a substantial degree of resistance to these microorganisms. In the present case, unwanted microorganisms are to be understood as meaning phytopathogenic fungi, bacteria and viruses. Thus, the substances according to the invention can be employed for protecting plants against attack by the abovementioned pathogens within a certain period of time after the treatment. The period of time within which their protection is effected generally extends from 1 to 10 days, preferably 1 to 7 days, after the treatment of the plants with the active compounds.

As already mentioned above, the active compound combinations according to the invention are effective against normally sensitive and resistant species and against all or individual stages of development of animal pests. The abovementioned pests include:

From the order of the Isopoda, for example, Oniscus asellus, Armadillidium vulgare, Porcellio scaber. From the order of the Diplopoda, for example, Blaniulus guttulatus. From the order of the Chilopoda, for example, Geophilus carpophagus, Scutigera spp. From the order of the Symphyla, for example, Scutigerella immaculata. From the order of the Thysanura, for example, Lepisma saccharina. From the order of the Collembola, for example, Onychiurus armatus. From the order of the Orthoptera, for example, Acheta domesticus, Gryllotalpa spp., Locusta migratoria migratorioides, Melanoplus spp., Schistocerca gregaria. From the order of the Blattaria, for example, Blatta orientalis, Periplaneta americana, Leucophaea maderae, Blattella germanica. From the order of the Dermaptera, for example, Forficula auricularia. From the order of the Isoptera, for example, Reticulitermes spp. From the order of the Phthiraptera, for example, Pediculus humanus corporis, Haematopinus spp., Linognathus spp., Trichodectes spp., Damalinia spp. From the order of the Thysanoptera, for example, Hercinothrips femoralis, Thrips tabaci, Thrips palmi, Frankliniella occidentalis. From the order of the Heteroptera, for example, Eurygaster spp., Dysdercus intermedius, Piesma quadrata, Cimex lectularius, Rhodnius prolixus, Triatoma spp. From the order of the Homoptera, for example, Aleurodes brassicae, Bemisia tabaci, Trialeurodes vaporariorum, Aphis gossypii, Brevicoryne brassicae, Cryptomyzus ribis, Aphis fabae, Aphis pomi, Eriosoma lanigerum, Hyalopterus arundinis, Phylloxera vastatrix, Pemphigus spp., Macro-siphum avenae, Myzus spp., Phorodon humuli, Rhopalosiphum padi, Empoasca spp., Euscelis bilobatus, Nephotettix cincticeps, Lecanium corni, Saissetia oleae, Laodelphax striatellus, Nilaparvata lugens, Aonidiella aurantii, Aspidiotus hederae, Pseudococcus spp., Psylla spp. From the order of the Lepidoptera, for example, Pectinophora gossypiella, Bupalus piniarius, Chematobia brumata, Lithocolletis blancardella, Hyponomeuta padella, Plutella xylostella, Malacosoma neustria, Euproctis chrysorrhoea, Lymantria spp., Bucculatrix thurberiella, Phyllocnistis citrella, Agrotis spp., Euxoa spp., Feltia spp., Earias insulana, Heliothis spp., Mamestra brassicae, Panolis flammea, Spodoptera spp., Trichoplusia ni, Carpocapsa pomonella, Pieris spp., Chilo spp., Pyrausta nubilalis, Ephestia kuehniella, Galleria mellonella, Tineola bisselliella, Tinea pellionella, Hofmannophila pseudospretella, Cacoecia podana, Capua reticulana, Choristoneura fumiferana, Clysia ambiguella, Homona magnanima, Tortrix viridana, Cnaphalocerus spp., Oulema oryzae. From the order of the Coleoptera, for example, Anobium punctatum, Rhizopertha dominica, Bruchidius obtectus, Acanthoscelides obtectus, Hylotrupes bajulus, Agelastica alni, Leptinotarsa decemlineata, Phaedon cochleariae, Diabrotica spp., Psylliodes chrysocephala, Epilachna varivestis, Atomaria spp., Oryzaephilus surinamensis, Anthonomus spp., Sitophilus spp., Otiorrhynchus sulcatus, Cosmopolites sordidus, Ceuthorrhynchus assimilis, Hypera postica, Dermestes spp., Trogoderma spp., Anthrenus spp., Attagenus spp., Lyctus spp., Meligethes aeneus, Ptinus spp., Niptus hololeucus, Gibbium psylloides, Tribolium spp., Tenebrio molitor, Agriotes spp., Conoderus spp., Melolontha melolontha, Amphimallon solstitialis, Costelytra zealandica, Lissorhoptrus oryzophilus. From the order of the Hymenoptera, for example, Diprion spp., Hoplocampa spp., Lasius spp., Monomorium pharaonis, Vespa spp. From the order of the Diptera, for example, Aedes spp., Anopheles spp., Culex spp., Drosophila melanogaster, Musca spp., Fannia spp., Calliphora erythrocephala, Lucilia spp., Chrysomyia spp., Cuterebra spp., Gastrophilus spp., Hyppobosca spp., Stomoxys spp., Oestrus spp., Hypoderma spp., Tabanus spp., Tannia spp., Bibio hortulanus, Oscinella frit, Phorbia spp., Pegomyia hyoscyami, Ceratitis capitata, Dacus oleae, Tipula paludosa, Hylemyia spp., Liriomyza spp. From the order of the Siphonaptera, for example, Xenopsylla cheopis, Ceratophyllus spp. From the class of arachnids, for example, Scorpio maurus, Latrodectus mactans, Acarus siro, Argas spp., Ornithodoros spp., Dermanyssus gallinae, Eriophyes ribis, Phyllocoptruta oleivora, Boophilus spp., Rhipicephalus spp., Amblyomma spp., Hyalomma spp., Ixodes spp., Psoroptes spp., Chorioptes spp., Sarcoptes spp., Tarsonemus spp., Bryobia praetiosa, Panonychus spp., Tetranychus spp., Hemitarsonemus spp., Brevipalpus spp.

The plant-parasitic nematodes include, for example, Pratylenchus spp., Radopholus similis, Ditylenchus dipsaci, Tylenchulus semipenetrans, Heterodera spp., Globodera spp., Meloidogyne spp., Aphelenchoides spp., Longidorus spp., Xiphinema spp., Trichodorus spp., Bursaphelenchus spp.

All plants and plant parts can be treated in accordance with the invention. Plants are understood as meaning, in the present context, all plants and plant populations, such as desired and undesired wild plants or crop plants (including naturally occurring crop plants). Crop plants may be plants which can be obtained by conventional breeding and optimization methods or by biotechnological and genetic engineering methods or by combinations of these methods, including the transgenic plants and including the plant varieties capable or not capable of being protected by plant breeders' rights. Plant parts are understood as meaning all above-ground and subterranean parts and organs of the plants, such as shoot, leaf, flower and root, examples which may be mentioned being leaves, needles, stalks, stems, flowers, fruiting bodies, fruits and seeds, and also roots, tubers and rhizomes. The plant parts also include harvested material and vegetative and generative propagation material, for example cuttings, tubers, rhizomes, slips and seeds.

What may be emphasized in this context is the particularly advantageous effect of the compositions according to the invention with regard to their use in cereal plants such as, for example, wheat, oats, barley, spelt, triticale and rye, but also in corn, sorghum and millet, rice, sugar cane, soybeans, sunflowers, potatoes, cotton, oilseed rape, canola, tobacco, sugar beet, fodder beet, asparagus, hops and fruit plants (comprising pome fruit such as, for example, apples and pears, stone fruit such as, for example, peaches, nectarines, cherries, plums and apricots, citrus fruits such as, for example, oranges, grapefruits, limes, lemons, kumquats, tangerines and satsumas, nuts such as, for example, pistachios, almonds, walnuts and pecan nuts, tropical fruits such as, for example, mango, paw-paw, pineapple, dates and bananas, and grapes) and vegetables (comprising leaf vegetables such as, for example, endives, corn salad, Florence fennel, lettuce, cos lettuce, Swiss chard, spinach and chicory for salad use, cabbages such as, for example, cauliflower, broccoli, Chinese leaves, Brassica oleracea (L.) convar. acephala var. sabellica L. (curly kale, feathered cabbage), kohlrabi, Brussels sprouts, red cabbage, white cabbage and savoy cabbage, fruit vegetables such as, for example, aubergines, cucumbers, capsicums, table pumpkins, tomatoes, courgettes and sweetcorn, root vegetables such as, for example celeriac, wild turnips, carrots, including yellow cultivars, Raphanus sativus var. niger and var. radicula, beetroot, scorzonera and celery, legumes such as, for example, peas and beans, and vegetables from the Allium family such as, for example, leeks and onions).

The treatment according to the invention of the plants and plant parts with the active compound combinations is carried out either directly or by treating their environment, habitat or storage space by the customary treatment methods, for example by dipping, spraying, vaporizing, misting, broadcasting, painting on and, in the case of propagation material, in particular seeds, furthermore by coating with one or more layers.

The mixtures according to the invention are particularly suitable for the treatment of seeds. Preferred combinations according to the invention are in this case the combinations mentioned above as being preferred or particularly preferred. Thus, most of the damage to crop plants which is caused by pests occurs as early as when the seed is infested during storage and after the seed is introduced into the soil, and during and immediately after germination of the plants. This phase is particularly critical since the roots and shoots of the growing plants are particularly sensitive and even minor damage can lead to the death of the whole plant. Protecting the seed and the germinating plant by the use of suitable compositions is therefore of particularly great interest.

The control of pests by treating the seed of plants has been known for a long time and is the subject of continuous improvements. However, the treatment of seed entails a series of problems which cannot always be solved in a satisfactory manner. Thus, it is desirable to develop methods for protecting the seed and the germinating plant which dispense with the additional application of crop protection products after planting or after emergence of the plants. It is furthermore desirable to optimize the amount of active compound employed in such a way as to provide optimum protection for the seed and the germinating plant from attack by pests, but without damaging the plant itself by the active compound employed. In particular, methods for the treatment of seed should also take into consideration the intrinsic insecticidal properties of transgenic plants in order to achieve optimum protection of the seed and the germinating plant with a minimum of crop protection products being employed.

The present invention therefore in particular also relates to a method for the protection of seed and of germinating plants from attack by pests, by treating the seed with a composition according to the invention. The invention likewise relates to the use of the compositions according to the invention for the treatment of seed for protecting the seed and the resulting plants from pests. Furthermore, the invention relates to seed which has been treated with a composition according to the invention so as to afford protection from pests.

One of the advantages of the present invention is that the particular systemic properties of the compositions according to the invention mean that treatment of the seed with these compositions not only protects the seed itself, but also the resulting plants after emergence, from pests. In this manner, the immediate treatment of the crop at the time of sowing or shortly thereafter can be dispensed with.

A further advantage is the synergistically increased insecticidal activity of the compositions according to the invention in comparison with the individual insecticidally active compound, which exceeds the total of the activity of the two active compounds when applied individually. Also advantageous is the synergistically increased fungicidal activity of the compositions according to the invention in comparison with the individual fungicidally active compound, which exceeds the expected activity of the active compound when applied individually. This makes possible an optimization of the amount of active compound employed.

Furthermore, it must be considered as advantageous that the mixtures according to the invention can also be employed in particular in transgenic seed, the plants arising from this seed being capable of expressing a protein directed against pests. By treating such seed with the compositions according to the invention, certain pests can be controlled merely by the expression of the, for example, insecticidal protein, and, additionally, the compositions according to the invention provide protection from damage.

The compositions according to the invention are suitable for protecting seed of any plant variety as already mentioned above which is employed in agriculture, in the greenhouse, in forests or horticulture. In particular, this takes the form of seed of corn, peanut, canola, oilseed rape, poppy, soybeans, cotton, beet (for example sugar beet and fodder beet), rice, sorghum and millet, wheat, barley, oats, rye, sunflower, tobacco, potato or vegetables (e.g. tomatoes, cabbage plants). The compositions according to the invention are likewise suitable for treating the seed of fruit plants and vegetables as already mentioned above. The treatment of the seed of corn, soybeans, cotton, wheat and canola or oilseed rape is of particular importance.

As already mentioned above, the treatment of transgenic seed with a composition according to the invention is also of particular importance. This takes the form of seed of plants which, as a rule, comprise at least one heterologous gene which governs the expression of a polypeptide with in particular insecticidal properties. In this context, the heterologous genes in transgenic seed may be derived from microorganisms such as Bacillus, Rhizobiuni, Pseudomonas, Serratia, Trichoderma, Clavibacter, Glomus or Gliocladium. The present invention is particularly suitable for the treatment of transgenic seed which comprises at least one heterologous gene originating from Bacillus sp. and whose gene product shows activity against the European corn borer and/or the corn root worm. It is particularly preferably a heterologous gene derived from Bacillus thuringiensis.

Within the scope of the present invention, the composition according to the invention is applied to the seed either alone or in suitable formulation. Preferably, the seed is treated in a state in which it is stable enough to avoid damage during treatment. In general, the seed may be treated at any point in time between harvest and sowing. The seed usually used has been separated from the plant and freed from cobs, shells, stalks, coats, hairs or the flesh of the fruits.

When treating the seed, care must generally be taken that the amount of the composition according to the invention applied to the seed and/or the amount of further additives is chosen in such a way that the germination of the seed is not adversely affected, or that the resulting plant is not damaged. This must be borne in mind in particular in the case of active compounds which can have phytotoxic effects at certain application rates.

The compositions according to the invention can be applied directly, that is to say without further components and without having been diluted. As a rule, it is preferable to apply the composition to the seed in the form of a suitable formulation. Suitable formulations and methods for the treatment of seed are known to the skilled worker and are described, for example, in the following documents: U.S. Pat. No. 4,272,417 A, U.S. Pat. No. 4,245,432 A, U.S. Pat. No. 4,808,430 A, U.S. Pat. No. 5,876,739 A, US 2003/0176428 A1, WO 2002/080675 A1, WO 2002/028186 A2.

The active compound combinations according to the invention can be converted into the customary formulations, such as solutions, emulsions, wettable powders, suspensions, powders, dusts, pastes, soluble powders, granules, suspoemulsion concentrates, natural and synthetic materials impregnated with active compounds and microencapsulations in polymeric materials.

These formulations are produced in a known manner, for example by mixing the active compounds with extenders, that is, liquid solvents and/or solid carriers, optionally with the use of surfactants, that is, emulsifiers and/or dispersants and/or foam formers.

If the extender used is water, it is also possible to use, for example, organic solvents as auxiliary solvents. Suitable liquid solvents are essentially: aromatics, such as xylene, toluene or alkylnaphthalenes, chlorinated aromatics or chlorinated aliphatic hydrocarbons, such as chlorobenzenes, chloroethylenes or methylene chloride, aliphatic hydrocarbons, such as cyclohexane or paraffins, for example mineral oil fractions, mineral and vegetable oils, alcohols, such as butanol or glycol and ethers and esters thereof, ketones, such as acetone, methyl ethyl ketone, methyl isobutyl ketone or cyclohexanone, strongly polar solvents, such as dimethylformamide and dimethyl sulfoxide, and water.

### Suitable Solid Carriers are:

for example ammonium salts and ground natural minerals, such as kaolins, clays, talc, chalk, quartz, attapulgite, montmorillonite or diatomaceous earth, and ground synthetic minerals, such as finely divided silica, alumina and silicates; suitable solid carriers for granules are: for example crushed and fractionated natural rocks such as calcite, marble, pumice, sepiolite and dolomite, and synthetic granules of inorganic and organic meals, and granules of organic material such as sawdust, coconut shells, corn cobs and tobacco stalks; suitable emulsifiers and/or foam formers are: for example nonionic and anionic emulsifiers, such as polyoxyethylene fatty acid esters, polyoxyethylene fatty alcohol ethers, for example alkylaryl polyglycol ethers, alkylsulfonates, alkyl sulfates, arylsulfonates and protein hydrolysates; suitable dispersants are: for example lignosulfite waste liquors and methylcellulose.

Tackifiers such as carboxymethylcellulose and natural and synthetic polymers in the form of powders, granules or latices, such as gum arabic, polyvinyl alcohol and polyvinyl acetate, and natural phospholipids, such as cephalins and lecithins, and synthetic phospholipids can be used in the formulations. Other possible additives are mineral and vegetable oils.

It is possible to use colorants such as inorganic pigments, for example iron oxide, titanium oxide and Prussian Blue, and organic dyestuffs, such as alizarin dyestuffs, azo dyestuffs and metal phthalocyanine dyestuffs, and trace nutrients such as salts of iron, manganese, boron, copper, cobalt, molybdenum and zinc.

The formulations in general comprise between 0.1 and 95% by weight of active compound, preferably between 0.5 and 90%.

Preferably, the active compound combinations according to the invention comprise no further active compounds apart from methiocarb and the neonicotinoids mentioned.

If appropriate, the active compound combinations according to the invention, in commercially available formulations and in the use forms prepared from these formulations, can be present in a mixture with other known active compounds such as insecticides, attractants, sterilants, bactericides, acaricides, nematicides, fungicides, growth regulators or herbicides. The insecticides include, for example, phosphoric esters, carbamates, carboxylic esters, chlorinated hydrocarbons, phenylureas, substances produced by microorganisms, and the like.

Examples of especially advantageous components in the mixtures are the following

### Fungicides:

aldimorph, ampropylfos, ampropylfos-potassium, andoprim, anilazin, azaconazole, azoxystrobin, benalaxyl, benodanil, benomyl, benzamacril, benzamacryl-isobutyl, bialaphos, binapacryl, biphenyl, bitertanol, blasticidin-S, bromuconazole, bupirimate, buthiobate, calcium polysulfide, capsimycin, captafol, captan, carbendazim, carboxin, carvone, quinomethionate, chlobenthiazon, chlorfenazole, chloroneb, chloropicrin, chlorothalonil, chlozolinate, clozylacon, cufraneb, cymoxanil, cyproconazole, cyprodinil, cyprofuram, debacarb, dichlorophen, diclobutrazol, diclofluanid, diclomezin, dicloran, diethofencarb, difenoconazole, dimethirimol, dimethomorph, diniconazole, diniconazole-M, dinocap, diphenylamine, dipyrithione, ditalimfos, dithianon, dodemorph, dodine, drazoxolon, ediphenphos, epoxiconazole, etaconazole, ethirimol, etridiazole, famoxadon, fenapanil, fenarimol, fenbuconazole, fenfuram, fenitropan, fenpiclonil, fenpropidin, fenpropimorph, fentin acetate, fentin hydroxide, ferbam, ferimzon, fluazinam, flumetover, fluoromid, fluquinconazole, flurprimidol, flusilazole, flusulfamide, flutolanil, flutriafol, folpet, fosetyl-aluminium, fosetyl-sodium, fthalide, fuberidazole, furalaxyl, furametpyr, furcarbonil, furconazole, furconazole-cis, furmecyclox, guazatine, hexachlorobenzene, hexaconazole, hymexazole, imazalil, imibenconazole, iminoctadine, iminoctadine-albesilate, iminoctadine-triacetate, iodocarb, ipconazole, iprobenfos (IBP), iprodione, irumamycin, isoprothiolane, isovaledione, kasugamycin, kresoxim-methyl, copper preparations such as: copper hydroxide, copper naphthenate, copper oxychloride, copper sulfate, copper oxide, oxine-copper and Bordeaux mixture, mancopper, mancozeb, maneb, meferimzone, mepanipyrim, mepronil, metalaxyl, metconazole, methasulfocarb, methfuroxam, metiram, metomeclam, metsulfovax, mildiomycin, myclobutanil, myclozolin, nickel dimethyldithiocarbamate, nitrothal-isopropyl, nuarimol, ofurace, oxadixyl, oxamocarb, oxolinic acid, oxycarboxim, oxyfenthiin, paclobutrazole, pefurazoate, penconazole, pencycuron, phosdiphen, pimaricin, piperalin, polyoxine, polyoxorim, probenazole, prochloraz, procymidon, propamocarb, propanosine-sodium, propiconazole, propineb, pyrazophos, pyrifenox, pyrimethanil, pyroquilon, pyroxyfur, quinconazole, quintozene (PCNB), sulfur and sulfur preparations, tebuconazole, tecloftalam, tecnazene, tetcyclacis, tetraconazole, thiabendazole, thicyofen, thifluzamide, thiophanate-methyl, thiram, tioxymid, tolclofos-methyl, tolylfluanid, triadimefon, triadimenol, triazbutil, triazoxide, trichlamide, tricyclazole, tridemorph, triflumizole, triforine, triticonazole, uniconazole, validamycin A, vinclozolin, viniconazole, zarilamid, zineb, ziram

and Dagger G, OK-8705, OK-8801, α-(1,1-dimethylethyl)-β-(2-phenoxyethyl)-1H-1,2,4-triazole-1-ethanol, α-(2,4-dichlorophenyl)-β-fluoro-β-propyl-1H-1,2,4-triazole-1-ethanol, α-(2,4-dichloro-phenyl)-β-methoxy-α-methyl-1H-1,2,4-triazole-1-ethanol, α-(5-methyl-1,3-dioxan-5-yl)-β-[[4-(tri-fluoromethyl)phenyl]methylene]-1H-1,2,4-triazole-1-ethanol, (5RS,6RS)-6-hydroxy-2,2,7,7-tetra-methyl-5-(1H-1,2,4-triazol-1-yl)-3-octanone, (E)-α-(methoxyimino)-N-methyl-2-phenoxyphenyl-acetamide, 1-isopropyl {2-methyl-1-[[[1-(4-methylphenyl)ethyl]amino]carbonyl]propyl}carbamate 1-(2,4-dichlorophenyl)-2-(1H-1,2,4-triazol-1-yl)ethanone O-(phenylmethyl)oxime, 1-(2-methyl-1-naphthalenyl)-1H-pyrrole-2,5-dione, 1-(3,5-dichlorophenyl)-3-(2-propenyl)-2,5-pyrrolidinedione, 1-[(diiodomethyl)sulfonyl]-4-methylbenzene, 1-[[2-(2,4-dichlorophenyl)-1,3-dioxolan-2-yl]methyl]-1H-imidazole, 1-[[2-(4-chlorophenyl)-3-phenyloxiranyl]methyl]-1H-1,2,4-triazole, 1-[1-[2-[(2,4-dichlorophenyl)methoxy]phenyl]ethenyl]-1H-imidazole, 1-methyl-5-nonyl-2-(phenylmethyl)-3-pyrrolidinol, 2′,6′-dibromo-2-methyl-4′-trifluoromethoxy-4′-trifluoromethyl-1,3-thiazole-5-carboxanilide, 2,2-dichloro-N-[1-(4-chlorophenyl)ethyl]-1-ethyl-3-methylcyclopropanecarboxamide, 2,6-dichloro-5-(methylthio)-4-pyrimidinyl thiocyanate, 2,6-dichloro-N-(4-trifluoromethylbenzyl)-benzamide, 2,6-dichloro-N-[[4-(trifluoromethyl)phenyl]methyl]benzamide, 2-(2,3,3-triiodo-2-propenyl)-2H-tetrazole, 2-[(i-methylethyl)sulfonyl]-5-(trichloromethyl)-1,3,4-thiadiazole, 2-[[6-deoxy-4-O-(4-O-methyl-β-D-glycopyranosyl)-Hc-D-glueopyranosyl]amino]-4-methoxy-1H-pyrrolo-[2,3-d]pyrimidine-5-carbonitrile, 2-aminobutane, 2-bromo-2-(bromomethyl)pentanedinitrile, 2-chloro-N-(2,3-dihydro-1,1,3-trimethyl-1H-inden-4-yl)-3-pyridinecarboxamide, 2-chloro-N-(2,6-dimethylphenyl)-N-(isothiocyanatomethyl)acetamide, 2-phenylphenol (OPP), 3,4-dichloro-1-[4-(difluoromethoxy)phenyl]-1H-pyrrole-2,5-dione, 3,5-dichloro-N—[cyano[(1-methyl-2-propynyl)oxy]-methyl]benzamide, 3-(1,1-dimethylpropyl-1-oxo)-1H-indene-2-carbonitrile, 3-[2-(4-chlorophenyl)-5-ethoxy-3-isoxazolidinyl]pyridine, 4-chloro-2-cyano-N,N-dimethyl-5-(4-methylphenyl)-1H-imidazole-1-sulfonamide, 4-methyltetrazolo[1,5-a]quinazolin-5 (4H)-one, 8-(1,1-dimethylethyl)-N-ethyl-N-propyl-1,4-dioxaspiro[4.5]decane-2-methanamine, 8-hydroxyquinoline sulfate, N²-[(phenyl-amino)carbonyl]-9H-xanthene-9-carbohydrazide, bis(1-methylethyl)-3-methyl-4-[(3-methylbenzoyl)-oxy]-2,5-thiophene dicarboxylate, cis-1-(4-chlorophenyl)-2-(1H-1,2,4-triazol-1-yl)cycloheptanol, cis-4-[3-[4-(1,1-dimethylpropyl)phenyl-2-methylpropyl]-2,6-dimethylmorpholine hydrochloride, ethyl [(4-chlorophenyl)azo]cyanoacetate, potassium hydrogencarbonate, sodium methanetetrathiolate, methyl 1-(2,3-dihydro-2,2-dimethyl-1H-inden-1-yl)-1H-imidazole-5-carboxylate, methyl N-(2,6-dimethylphenyl)-N-(5-isoxazolylcarbonyl)-DL-alaninate, methyl N-(chloroacetyl)-N-(2,6-dimethyl-phenyl)-DL-alaninate, N-(2,3-dichloro-4-hydroxyphenyl)-1-methylcyclohexanecarboxamide, N-(2,6-dimethylphenyl)-2-methoxy-N-(tetrahydro-2-oxo-3-furanyl)acetamide, N-(2,6-dimethylphenyl)-2-methoxy-N-(tetrahydro-2-oxo-3-thienyl)acetamide, N-(2-chloro-4-nitrophenyl)-4-methyl-3-nitrobenzenesulfonamide, N-(4-cyclohexylphenyl)-1,4,5,6-tetrahydro-2-pyrimidineamine, N-(4-hexylphenyl)-1,4,5,6-tetrahydro-2-pyrimidineamine, N-(5-chloro-2-methylphenyl)-2-methoxy-N-(2-oxo-3-oxazolidinyl)acetamide, N-((6-methoxy)-3-pyridinyl)cyclopropanecarboxamide, N-[2,2,2-tri-chloro-1-[(chloroacetyl)amino]ethyl]benzamide, N-[3-chloro-4,5-bis(2-propynyloxy)phenyl]-N′-methoxymethaneimidamide, sodium N-formyl-N-hydroxy-DL-alaninate, O,O-diethyl[2-(dipropyl-amino)-2-oxoethyl]ethylphosphoramidothioate, O-methyl S-phenyl phenylpropylphosphoramidothioate, S-methyl 1,2,3-benzothiadiazole-7-carbothioate, spiro[2H]-1-benzopyran-2,1′(3′H)-iso-benzofuran]-3′-one.

### Bactericides:

bronopol, dichlorophen, nitrapyrin, nickel dimethyldithiocarbamate, kasugamycin, octhilinone, furancarboxylic acid, oxytetracyclin, probenazole, streptomycin, tecloftalam, copper sulfate and other copper preparations.

### Insecticides/Acaricides/Nematicides:

abamectin, ABG-9008, acephate, acequinocyl, acetamiprid, acetoprole, acrinathrin, AKD-1022, AKD-3059, AKD-3088, alanycarb, aldicarb, aldoxycarb, allethrin, alpha-cypermethrin (alphamethrin), amidoflumet, aminocarb, amitraz, avermectin, AZ-60541, azadirachtin, azamethiphos, azinphos-methyl, azinphos-ethyl, azocyclotin, Bacillus popilliae, Bacillus sphaericus, Bacillus subtilis, Bacillus thuringiensis, Bacillus thuringiensis strain EG-2348, Bacillus thuringiensis strain GC-91, Bacillus thuringiensis strain NCTC-11821, baculoviruses, Beauveria bassiana, Beauveria tenella, benclothiaz, bendiocarb, benfuracarb, bensultap, benzoximate, beta-cyfluthrin, beta-cypermethrin, bifenazate, bifenthrin, binapacryl, bioallethrin, bioallethrin-5-cyclopentyl-isomer, bioethanomethrin, biopermethrin, bioresmethrin, bistrifluoron, BPMC, brofenprox, bromophos-ethyl, bromopropylate, bromfenvinfos (-methyl), BTG-504, BTG-505, bufencarb, buprofezin, butathiofos, butocarboxim, butoxycarboxim, butylpyridaben, cadusafos, camphechlor, carbaryl, carbofuran, carbophenothion, carbosulfan, cartap, CGA-50439, chinomethionat, chlordane, chlordimeform, chloethocarb, chlorethoxyfos, chlorfenapyr, chlorfenvinphos, chlorfluazuron, chlormephos, chlorobenzilate, chloropicrin, chlorproxyfen, chlorpyrifos-methyl, chlorpyrifos (-ethyl), chlovaporthrin, chromafenozide, cis-cypermethrin, cis-resmethrin, cis-permethrin, clocythrin, cloethocarb, clofentezine, clothianidin, clothiazoben, codlemone, coumaphos, cyanofenphos, cyanophos, cycloprene, cycloprothrin, cyfluthrin, cyflumetofen, cyhalothrin, cyhexatin, cypermethrin, cyphenothrin (IR-trans-isomer), cyromazine, DDT, deltamethrin, demeton-5-methyl, demeton-5-methylsulfone, diafenthiuron, dialifos, diazinon, dichlofenthion, dichlorvos, dicofol, dicrotophos, dicyclanil, diflubenzuron, dimefluthrin, dimethoate, dimethylvinphos, dinobuton, dinocap, dinotefuran, diofenolan, disulfoton, docusat-sodium, dofenapyn, DOWCO-439, eflusilanate, emamectin, emamectin-benzoate, empenthrin (IR-isomer), endosulfan, Entomopthora spp., EPN, esfenvalerate, ethiofencarb, ethion, ethiprole, ethoprophos, etofenprox, etoxazole, etrimfos, famphur, fenamiphos, fenazaquin, fenbutatin oxide, fenfluthrin, fenitrothion, fenobucarb, fenothiocarb, fenoxacrim, fenoxycarb, fenpropathrin, fenpyrad, fenpyrithrin, fenpyroximate, fensulfothion, fenthion, fentrifanil, fenvalerate, fipronil, flonicamid, fluacrypyrim, fluazuron, flubendiamide, flubenzimine, flubrocythrinate, flucycloxuron, flucythrinate, flufenerim, flufenoxuron, flufenprox, flumethrin, flupyrazofos, flutenzin (flufenzine), fluvalinate, fonofos, formetanate, formothion, fosmethilan, fosthiazate, fubfenprox (fluproxyfen), furathiocarb, gamma-cyhalothrin, gamma-HCH, gossyplure, grandlure, granulosis viruses, halfenprox, halofenozide, HCH, HCN-801, heptenophos, hexaflumuron, hexythiazox, hydramethylnone, hydroprene, IKA-2002, imidacloprid, imiprothrin, indoxacarb, iodofenphos, iprobenfos, isazofos, isofenphos, isoprocarb, isoxathion, ivermectin, japonilure, kadethrin, nuclear polyhedrosis viruses, kinoprene, lambda-cyhalothrin, lindane, lufenuron, malathion, mecarbam, mesulfenfos, metaldehyde, metam-sodium, methacrifos, methamidophos, Metharhizium anisopliae, Metharhizium flavoviride, methidathion, methiocarb, methomyl, methoprene, methoxychlor, methoxyfenozide, metofluthrin, metolcarb, metoxadiazone, mevinphos, milbemectin, milbemycin, MKI-245, MON-45700, monocrotophos, moxidectin, MTI-800, naled, NC-104, NC-170, NC-184, NC-194, NC-196, niclosamide, nicotine, nitenpyram, nithiazine, NNI-0001, NNI-0101, NNI-0250, NNI-9768, novaluron, noviflumuron, OK-5101, OK-5201, OK-9601, OK-9602, OK-9701, OK-9802, omethoate, oxamyl, oxydemeton-methyl, Paecilomyces fumosoroseus, parathion-methyl, parathion (-ethyl), permethrin (cis-, trans-), petroleum, PH-6045, phenothrin (IR-trans isomer), phenthoate, phorate, phosalone, phosmet, phosphamidon, phosphocarb, phoxim, piperonyl butoxide, pirimicarb, pirimiphos-methyl, pirimiphos-ethyl, potassium oleate, prallethrin, profenofos, profluthrin, promecarb, propaphos, propargite, propetamphos, propoxur, prothiofos, prothoate, protrifenbute, pymetrozine, pyraclofos, pyrafluprole, pyresmethrin, pyrethrum, pyridaben, pyridalyl, pyridaphenthion, pyridathion, pyrimidifen, pyriprole, pyriproxyfen, quinalphos, resmethrin, RH-5849, ribavirin, RU-12457, RU-15525, S-421, S-1833, salithion, sebufos, SI-0009, silafluofen, spinosad, spirodiclofen, spiromesifen, sulfluramid, sulfotep, sulprofos, SZI-121, tau-fluvalinate, tebufenozide, tebufenpyrad, tebupirimfos, teflubenzuron, tefluthrin, temephos, temivinphos, terbam, terbufos, tetrachlorvinphos, tetradifon, tetramethrin, tetramethrin (IR-isomer), tetrasul, theta-cypermethrin, thiacloprid, thiamethoxam, thiapronil, thiatriphos, thiocyclam hydrogenoxalate, thiodicarb, thiofanox, thiometon, thiosultap-sodium, thuringiensin, tolfenpyrad, tralocythrin, tralomethrin, transfluthrin, triarathene, triazamate, triazophos, triazuron, trichlophenidine, trichlorfon, triflumuron, trimethacarb, vamidothion, vaniliprole, verbutin, Verticillium lecanii, WL-108477, WL-40027, yl-5201, yl-5301, yl-5302, XMC, xylylcarb, ZA-3274, zeta-cypermethrin, zolaprofos, ZXI-8901, the compound 3-methylphenyl propylcarbamate (Tsumacide Z), the compound 3-(5-chloro-3-pyridinyl)-8-(2,2,2-trifluoroethyl)-8-azabicyclo[3.2.1]octane-3-carbonitrile (CAS-Reg. No. 185982-80-3) and the corresponding 3-endo-isomer (CAS-Reg. No. 185984-60-5) (cf. WO-96/37494, WO-98/25923), and also preparations which comprise insecticidally active plant extracts, nematodes, fungi or viruses.

A mixture with other known active compounds, such as herbicides, or with fertilizers and growth regulators is also possible.

When used as insecticides, the active compound combinations according to the invention can furthermore be present in their commercially available formulations and in the use forms, prepared from these formulations, as a mixture with synergistic agents. Synergistic agents are compounds which increase the action of the active compounds, without it being necessary for the synergistic agent added to be active itself.

The active compound content of the use forms prepared from the commercially available formulations can vary within wide limits. The active compound concentration of the use forms can be from 0.0000001 to 95% by weight of active compound, preferably between 0.0001 and 1% by weight.

The compounds are employed in a customary manner appropriate for the use forms.

When used against hygiene pests and pests of stored products, the active compound combinations have an excellent residual action on wood and clay as well as a good stability to alkali on limed substrates.

Industrial materials in the present context are understood as meaning non-live materials such as, preferably, plastics, adhesives, sizes, paper and board, leather, wood, timber products and paints.

The material which is to be protected from insect attack is very especially preferably wood and timber products.

Wood and timber products which can be protected by the composition according to the invention, or mixtures comprising it, are to be understood as meaning, for example:

Construction timber, wooden beams, railway sleepers, bridge components, jetties, vehicles made of wood, boxes, pallets, containers, telephone poles, wood lagging, windows and doors made of wood, plywood, chipboard, joinery, or timber products which quite generally are used in house construction or building joinery.

The active compound combinations can be used as such, in the form of concentrates or generally customary formulations such as powders, granules, solutions, suspensions, emulsions or pastes.

The abovementioned formulations can be prepared in a manner known per se, for example by mixing the active compounds with at least one solvent or diluent, emulsifier, dispersant and/or binder or fixative, water repellant, if desired desiccants and UV stabilizers, and if desired colorants and pigments and other processing auxiliaries.

The insecticidal compositions or concentrates used for protecting wood and timber products comprise the active compound according to the invention in a concentration of from 0.0001 to 95% by weight, in particular 0.001 to 60% by weight.

The amount of the compositions or concentrates employed depends on the species and the abundance of the insects and on the medium. The optimal quantity to be employed can be determined in each case by test series upon application. In general, however, it will suffice to employ from 0.0001 to 20% by weight, preferably 0.001 to 10% by weight, of the active compound, based on the material to be protected.

A suitable solvent and/or diluent is an organochemical solvent or solvent mixture and/or an oily or oil-type organochemical solvent or solvent mixture of low volatility and/or a polar organochemical solvent or solvent mixture and/or water and, if appropriate, an emulsifier and/or wetter.

Organochemical solvents which are preferably employed are oily or oil-type solvents with an evaporation number of above 35 and a flash point of above 30° C., preferably above 45° C. Such oily and oil-type solvents which are insoluble in water and of low volatility and which are used are suitable mineral oils or their aromatic fractions or mineral-oil-containing solvent mixtures, preferably white spirit, petroleum and/or alkylbenzene.

Mineral oils which are advantageously used are those with a boiling range of from 170 to 220° C., white spirit with a boiling range of from 170 to 220° C., spindle oil with a boiling range of from 250 to 350° C., petroleum and aromatics with a boiling range of from 160 to 280° C., oil of turpentine, and the like.

In a preferred embodiment, liquid aliphatic hydrocarbons with a boiling range of from 180 to 210° C. or high-boiling mixtures of aromatic and aliphatic hydrocarbons with a boiling range of from 180 to 220° C. and/or spindle oil and/or monochloronaphthalene, preferably α-monochloro-naphthalene, are used.

The organic oily or oil-type solvents of low volatility and with an evaporation number of above 35 and a flash point of above 30° C., preferably above 45° C., can be replaced in part by organochemical solvents of high or medium volatility, with the proviso that the solvent mixture likewise has an evaporation number of above 35 and a flash point of above 30° C., preferably above 45° C., and that the insecticide/fungicide mixture is soluble or emulsifiable in this solvent mixture.

In a preferred embodiment, some of the organochemical solvent or solvent mixture is replaced by an aliphatic polar organochemical solvent or solvent mixture. Aliphatic organochemical solvents which contain hydroxyl and/or ester and/or ether groups are preferably used, such as, for example, glycol ethers, esters or the like.

Organochemical binders used for the purposes of the present invention are the synthetic resins and/or binding drying oils which are known per se and which can be diluted in water and/or dissolved or dispersed or emulsified in the organochemical solvents employed, in particular binders composed of, or comprising, an acrylate resin, a vinyl resin, for example polyvinyl acetate, polyester resin, polycondensation or polyaddition resin, polyurethane resin, alkyd resin or modified alkyd resin, phenol resin, hydrocarbon resin such as indene/coumarone resin, silicone resin, drying vegetable and/or drying oils and/or physically drying binders based on a natural and/or synthetic resin.

The synthetic resin employed as binder can be employed in the form of an emulsion, dispersion or solution. Bitumen or bituminous substances may also be used as binders, in amounts of up to 10% by weight. In addition, colorants, pigments, water repellants, odor-masking agents, and inhibitors or anticorrosive agents and the like, all of which are known per se, can be employed.

In accordance with the invention, the composition or the concentrate preferably comprises, as organochemical binders, at least one alkyd resin or modified alkyd resin and/or a drying vegetable oil. Alkyd resins which are preferably used in accordance with the invention are those with an oil content of over 45% by weight, preferably 50 to 68% by weight.

Some or all of the abovementioned binder can be replaced by a fixative (mixture) or plasticizer (mixture). These additives are intended to prevent volatilization of the active compounds, and also crystallization or precipitation. They preferably replace from 0.01 to 30% of the binder (based on 100% of binder employed).

The plasticizers are from the chemical classes of the phthalic esters, such as dibutyl phthalate, dioctyl phthalate or benzyl butyl phthalate, phosphoric esters such as tributyl phosphate, adipic esters such as di-(2-ethylhexyl) adipate, stearates such as butyl stearate or amyl stearate, oleates such as butyl oleate, glycerol ethers or higher-molecular-weight glycol ethers, glycerol esters and p-toluenesulfonic esters.

Fixatives are based chemically on polyvinyl alkyl ethers such as, for example, polyvinyl methyl ether, or ketones such as benzophenone and ethylenebenzophenone.

Other suitable solvents or diluents are, in particular, also water, if appropriate as a mixture with one or more of the abovementioned organochemical solvents or diluents, emulsifiers and dispersants.

Particularly effective timber protection is achieved by industrial-scale impregnating processes, for example the vacuum, double-vacuum or pressure processes.

If appropriate, the ready-to-use compositions may additionally also contain further insecticides and also, if appropriate, one or more fungicides.

The active compound combinations according to the invention can at the same time be employed for protecting objects which come into contact with seawater or brackish water, in particular hulls, screens, nets, buildings, moorings and signaling systems, from fouling.

Fouling by sessile Oligochaeta, such as Serpulidae, and by shells and species from the Ledamorpha group (goose barnacles), such as various Lepas and Scalpellum species, or by species from the Balanomorpha group (acorn barnacles), such as Balanus or Pollicipes species, increases the frictional drag of ships and, as a consequence, leads to a marked increase in operation costs owing to higher energy consumption and additionally frequent residences in the dry dock.

Apart from fouling by algae, for example Ectocarpus sp. and Ceramium sp., fouling by sessile Entomostraka groups, which come under the generic term Cirripedia (cirriped crustaceans), is of particular importance.

Surprisingly, it has now been found that the active compound combinations according to the invention have an outstanding antifouling action.

Use of the active compound combinations according to the invention allows the use of heavy metals such as, for example, in bis(trialkyltin) sulfides, tri-n-butyltin laurate, tri-n-butyltin chloride, copper(I) oxide, triethyltin chloride, tri-n-butyl(2-phenyl-4-chlorophenoxy)tin, tributyltin oxide, molybdenum disulfide, antimony oxide, polymeric butyl titanate, phenyl(bispyridine)bismuth chloride, tri-n-butyltin fluoride, manganese ethylenebisthiocarbamate, zinc dimethyldithiocarbamate, zinc ethylenebisthiocarbamate, zinc salts and copper salts of 2-pyridinethiol 1-oxide, bisdimethyldithiocarbamoylzinc ethylenebisthiocarbamate, zinc oxide, copper(I) ethylenebisdithiocarbamate, copper thiocyanate, copper naphthenate and tributyltin halides to be dispensed with, or the concentration of these compounds to be substantially reduced.

If appropriate, the ready-to-use antifouling paints can additionally comprise other active compounds, preferably algicides, fungicides, herbicides, molluscicides, or other antifouling active compounds.

Preferable suitable components in combinations with the antifouling compositions according to the invention are:

algicides such as 2-tert-butylamino-4-cyclopropylamino-6-methylthio-1,3,5-triazine, dichlorophen, diuron, endothal, fentin acetate, isoproturon, methabenzthiazuron, oxyfluorfen, quinoclamine and terbutryn;
fungicides such as benzo[b]thiophenecarboxylic acid cyclohexylamide S,S-dioxide, dichlofluanid, fluorfolpet, 3-iodo-2-propynyl butylcarbamate, tolylfluanid and azoles such as azaconazole, cyproconazole, epoxyconazole, hexaconazole, metconazole, propiconazole and tebuconazole;
molluscicides such as Fe complexing agents, fentin acetate, metaldehyde, methiocarb, niclosamid, thiodicarb and trimethacarb;
or conventional antifouling active compounds such as 4,5-dichloro-2-octyl-4-isothiazolin-3-one, diiodomethylparatryl sulfone, 2-(N,N-dimethylthiocarbamoylthio)-5-nitrothiazyl, potassium salts, copper salts, sodium salts and zinc salts of 2-pyridinethiol 1-oxide, pyridine-triphenylborane, tetrabutyldistannoxane, 2,3,5,6-tetrachloro-4-(methylsulfonyl)pyridine, 2,4,5,6-tetrachloro-isophthalonitrile, tetramethylthiuram disulfide and 2,4,6-trichlorophenylmaleimide.

The antifouling compositions used comprise the active compound in a concentration of 0.001 to 50% by weight, in particular 0.01 to 20% by weight.

Moreover, the antifouling compositions comprise the customary components such as, for example, those described in Ungerer, Chem. Ind. 1985, 37, 730-732 and Williams, Antifouling Marine Coatings, Noyes, Park Ridge, 1973.

Besides the algicidal, fungicidal, molluscicidal and insecticidal active compounds, antifouling paints comprise, in particular, binders.

Examples of recognized binders are polyvinyl chloride in a solvent system, chlorinated rubber in a solvent system, acrylic resins in a solvent system, in particular in an aqueous system, vinyl chloride/vinyl acetate copolymer systems in the form of aqueous dispersions or in the form of organic solvent systems, butadiene/styrene/acrylonitrile rubbers, drying oils such as linseed oil, resin esters or modified hardened resins in combination with tar or bitumens, asphalt and epoxy compounds, small amounts of chlorine rubber, chlorinated polypropylene and vinyl resins.

If appropriate, paints also comprise inorganic pigments, organic pigments or colorants which are preferably insoluble in seawater. Paints may furthermore comprise materials such as colophonium to allow controlled release of the active compounds. Furthermore, the paints may comprise plasticizers, modifiers which affect the rheological properties and other conventional constituents.

The active compound combinations according to the invention may also be incorporated into self-polishing antifouling systems.

The active compound combinations according to the invention are also suitable for controlling animal pests, in particular insects, arachnids and mites, which are found in enclosed spaces such as, for example, dwellings, factory halls, offices, vehicle cabins and the like. They can be employed alone or in combination with other active compounds and auxiliaries in domestic insecticide products for controlling these pests. They are active against sensitive and resistant species and against all developmental stages. These pests include:

From the order of the Scorpionidea, for example, Buthus occitanus. From the order of the Acarina, for example, Argas persicus, Argas reflexus, Bryobia ssp., Dermanyssus gallinae, Glyciphagus domesticus, Omithodorus moubat, Rhipicephalus sanguineus, Trombicula alfreddugesi, Neutrombicula autumnalis, Dermatophagoides pteronissimus, Dennatophagoides forinae. From the order of the Araneae, for example, Aviculariidae, Araneidae. From the order of the Opiliones, for example, Pseudoscorpiones chelifer, Pseudoscorpiones cheiridium, Opiliones phalangium. From the order of the Isopoda, for example, Oniscus asellus, Porcellio scaber. From the order of the Diplopoda, for example, Blaniulus guttulatus, Polydesmus spp. From the order of the Chilopoda, for example, Geophilus spp. From the order of the Zygentoma, for example, Ctenolepisma spp., Lepisma saccharina, Lepismodes inquilinus. From the order of the Blattaria, for example, Blatta orientalies, Blattella germanica, Blattella asahinai, Leucophaea maderae, Panchlora spp., Parcoblatta spp., Periplaneta australasiae, Periplaneta americana, Periplaneta brunnea, Periplaneta fuliginosa, Supella longipalpa. From the order of the Saltatoria, for example, Acheta domesticus. From the order of the Dermaptera, for example, Forficula auricularia. From the order of the Isoptera, for example, Kalotermes spp., Reticulitermes spp. From the order of the Psocoptera, for example, Lepinatus spp., Liposcelis spp. From the order of the Coleptera, for example, Anthrenus spp., Attagenus spp., Dermestes spp., Latheticus oryzae, Necrobia spp., Ptinus spp., Rhizopertha dominica, Sitophilus granarius, Sitophilus oryzae, Sitophilus zeamais, Stegobium paniceum. From the order of the Diptera, for example, Aedes aegypti, Aedes albopictus, Aedes taeniorhynchus, Anopheles spp., Calliphora erythrocephala, Chrysozona pluvialis, Culex quinquefasciatus, Culex pipiens, Culex tarsalis, Drosophila spp., Fannia canicularis, Musca domestica, Phlebotomus spp., Sarcophaga carnaria, Simulium spp., Stomoxys calcitrans, Tipula paludosa. From the order of the Lepidoptera, for example, Achroia grisella, Galleria mellonella, Plodia interpunctella, Tinea cloacella, Tinea pellionella, Tineola bisselliella. From the order of the Siphonaptera, for example, Ctenocephalides canis, Ctenocephalides felis, Pulex irritans, Tunga penetrans, Xenopsylla cheopis. From the order of the Hymenoptera, for example, Camponotus herculeanus, Lasius fuliginosus, Lasius niger, Lasius umbratus, Monomorium pharaonis, Paravespula spp., Tetramorium caespitum. From the order of the Anoplura, for example, Pediculus humanus capitis, Pediculus humanus corporis, Phthirus pubis. From the order of the Heteroptera, for example, Cimex hemipterus, Cimex lectularius, Rhodnius prolixus, Triatoma infestans.

Application in the field of the household insecticides can also be effected in combination with other suitable active compounds such as phosphoric esters, carbamates, pyrethroids, growth regulators or active compounds from other known classes of insecticides.

They are used in aerosols, pressure-free spray products, for example pump and atomizer sprays, automatic fogging systems, foggers, foams, gels, evaporator products with evaporator tablets made of cellulose or polymer, liquid evaporators, gel and membrane evaporators, propeller-driven evaporators, energy-free, or passive, evaporation systems, moth papers, moth bags and moth gels, as granules or dusts, in baits for spreading or in bait stations.

When applying the active compound combinations according to the invention, the application rates can be varied within a substantial range, depending on the type of application. In the treatment of plant parts, the application rates of active compound combinations are generally between 0.1 and 10 000 g/ha, preferably between 10 and 1000 g/ha.

The good insecticidal action of the active compound combinations according to the invention can be seen from the examples which follow. While the individual active compounds exhibit weaknesses with regard to the action, the combinations demonstrate an action which exceeds the simple summation of action.

Formula for the calculation of the kill rate of a combination of two active compounds

The expected activity for a given combination of two active compounds can be calculated (cf. COLBY, S. R.; “Calculating Synergistic and Antagonistic Responses of Herbicide Combinations”, Weeds 15, pages 20-22, 1967):

if
X=the kill rate, expressed in % of the untreated control, when employing active compound A at an application rate of m ppm,
Y=the kill rate, expressed in % of the untreated control, when employing active compound B at an application rate of n ppm,
E=the kill rate, expressed in % of the untreated control, when employing active compounds A and B at application rates of m and n ppm,
   then

$$E = X + Y - \frac{X \times Y}{100}$$

If the actual insecticidal kill rate is higher than the calculated one, the kill rates of the combination are superadditive, i.e. a synergistic effect is present. In this case, the kill rate that is actually observed has to be higher than the value, calculated using the formula above, for the expected kill rate (E).

### EXAMPLE A

### Myzus persicae Test

| | |
|---|---|
| Solvent: | 7 parts by weight of dimethylformamide |
| Emulsifier: | 2 parts by weight of alkylaryl polyglycol ether |

To produce a suitable preparation of active compound, 1 part by weight of active compound is mixed with the stated amounts of solvent and emulsifier, and the concentrate is diluted with emulsifier-containing water to the desired concentration.

Cabbage leaves (Brassica olerace) which are heavily infested by the green peach aphid (Myzits persicae) are treated by being dipped into the preparation of active compound of the desired concentration.

After the desired period of time, the kill in % is determined. 100% means that all aphids have been killed; 0% means that none of the aphids have been killed.

The determined kill rates are entered into Colby's formula.

In this test, for example, the following active compound combination according to the present application shows a synergistically enhanced activity compared to the active compounds applied individually:

TABLE A1
Plant-damaging insects
Myzus persicae test

| | Concentration | Kill | |
|---|---|---|---|
| Active compound | in ppm | in % after 1 day | |
| clothianidin | 0.8 | 40 | |
| picoxystrobin | 100 | 0 | |
| | | found* | calc.** |
| clothianidin + picoxystrobin | 0.8 + 100 | 80 | 40 |
| (1:125) | | | |

*found = activity found
**calc. = activity calculated using Colby's formula

TABLE A2
Plant-damaging insects
Myzus persicae test

| | Concentration | Kill | |
|---|---|---|---|
| Active compound | in ppm | in % after 6 days | |
| clothianidin | 0.8 | 30 | |
| pyraclostrobin | 100 | 35 | |
| | | found* | calc.** |
| clothianidin + picoxystrobin | 0.8 + 100 | 85 | 54.5 |
| (1:125) | | | |

*found = activity found
**calc. = activity calculated using Colby's formula

### EXAMPLE B

### Phaedon cochleariae Larvae Test

| | |
|---|---|
| Solvent: | 7 parts by weight of dimethylformamide |
| Emulsifier: | 2 parts by weight of alkylaryl polyglycol ether |

To produce a suitable preparation of active compound, 1 part by weight of active compound is mixed with the stated amounts of solvent and emulsifier, and the concentrate is diluted with emulsifier-containing water to the desired concentration.

Cabbage leaves (Brassica oleracea) are treated by being dipped into the preparation of active compound of the desired concentration and populated with larvae of the mustard beetle (Phaedon cochleariae) while the leaves are still moist.

After the desired period of time, the kill in % is determined. 100% means that all beetle larvae have been killed; 0% means that none of the beetle larvae have been killed. The determined kill rates are entered into Colby's formunLla (see sheet 1).

In this test, the following active compound combination in accordance with the present application shows a synergistically enhanced activity compared to the active compounds applied individually:

TABLE B1
Plant-damaging insects
Phaedon cochleariae larvae test

| | Concentration | Kill | |
|---|---|---|---|
| Active compound/product | in ppm | in % after 6^{d} | |
| clothianidin | 4 | 20 | |
| picoxystrobin | 100 | 0 | |
| | | found* | calc.** |
| clothianidin + picoxystrobin | 4 + 100 | 70 | 20 |
| (1:25) | | | |

*found = activity found
**calc. = activity calculated using Colby's formula

TABLE B2
Plant-damaging insects
Phaedon cochleariae larvae test

| | Concentration | Kill | |
|---|---|---|---|
| Active compound/product | in ppm | in % after 6^{d} | |
| clothianidin | 4 | 60 | |
| dimoxystrobin | 20 | 0 | |
| | | found* | calc.** |
| clothianidin + picoxystrobin | 4 + 20 | 90 | 60 |
| (1:5) | | | |

*found = activity found
**calc. = activity calculated using Colby's formula

1. A composition comprising a synergistically effective mixture of
(a) at least one compound selected from the group consisting of clothianidin, imidacloprid, thiacloprid, dinotefuran, acetamiprid, nitenpyram and thiamethoxam, and
(b) at least one compound selected from the group consisting of picoxystrobin, pyraclostrobin, dimoxystrobin, metominostrobin and orysastrobin.

2. The composition as claimed in claim 1, wherein the compound of group (a) is clothianidin.

3. The composition as claimed in claim 1, wherein the compound of group (a) is imidacloprid.

4. The composition as claimed in claim 1, wherein compound of group (a) is thiacloprid.

5. The composition as claimed in claim 1, wherein the weight ratio of the compound of group (a) to the compound of group (b) is between 1000:1 and 1:100.

6. A method of treating a seed, comprising contacting said seed with a composition as claimed in claim 1.

7. A method of controlling pests which attack plants or seeds, comprising contacting said pests or their habitats with a composition as claimed in claim 1.

8. A method of controlling fungi which attack plants or seeds, comprising contacting said fungi with a composition as claimed as claim 1.

9. (canceled)

10. (canceled)

11. A process for preparing a pesticide, comprising mixing the synergistically effective mixture as claimed in claim 1 with one or more extenders, one or more surfactants, or a combination of one or more extenders and one or more surfactants.

12. A seed treated with a composition as claimed in claim 1.
